(19) United States
(12) Patent Application Publication
(10) Pub. No.: US 2007/0166381 A1
(43) Pub. Date: Jul. 19, 2007

(54) OSMOTIC DOSAGE FORM WITH CONTROLLED RELEASE AND FAST RELEASE ASPECTS

(76) Inventors: Iran Reyes, San Jose, CA (US); Julie Lee, Sunnyvale, CA (US); Brian Barclay, Sunnyvale, CA (US); Iswadi Liejanto, Redwood City, CA (US); Nipun Davar, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/619,468

(22) Filed: Jan. 3, 2007

Related U.S. Application Data
(60) Provisional application No. 60/756,766, filed on Jan. 6, 2006.

(51) Int. Cl. A61K 9/24

(52) U.S. Cl. 424/473

(57) ABSTRACT

Disclosed are osmotic dosage forms and methods that provide for fast release of drugs together with controlled release of drugs. In an aspect, disclosed are osmotic dosage forms including: a semi-permeable membrane; a lubricating subcoat located within the semi-permeable membrane; an orifice in the semi-permeable membrane located at an end of the semi-permeable membrane; a drug layer located adjacent to the orifice and within the lubricating subcoat; a push layer located within the lubricating subcoat and on a side of the drug layer opposite from the orifice; wherein an area of the orifice is greater than or equal to about 1,600 mil²; and wherein the drug layer comprises from about 20 wt % to about 90 wt % microcrystalline cellulose, and less than or equal to about 10 wt % of a drug, based on the total weight of the drug layer. Also disclosed are methods of making osmotic dosage forms.

### CROSS REFERENCE TO RELATED U.S. APPLICATION DATA

The present application is derived from and claims priority to provisional application U.S. Ser. No. 60/756,766, filed Jan. 6, 2006, which is herein incorporated by reference in its entirety.

### FIELD OF THE INVENTION

The invention pertains to osmotic dosage forms having a controlled release drug layer and a fast release drug layer, and related methods.

### BACKGROUND

Osmotic dosage forms for controlled delivery of drugs have been known in the art for a number of years. In certain circumstances, it is desirable to combine fast release of a drug together with controlled release of a same or different drug from the same dosage form.

For instance, U.S. Pat. Nos. 4,814,181; 4,915,953; 5,240,713; and 4,915,954 describe osmotic controlled release dosage forms that comprise a first lamina that is delivered in a short period of time and a second lamina that is delivered in a prolonged period of time. However, these osmotic dosage forms do not provide for a push layer that can promote better delivery control.

U.S. Pat. Nos. 6,919,373 and 6,930,129 disclose use of an immediate release overcoat for the delivery of methylphenidate from osmotic dosage forms. However, overcoating an osmotic dosage form can add cost, and reduce delivery control of the immediately release component.

U.S. Pat. Nos. 6,387,403, and 6,630,165 disclose use of a barrier layer in an osmotic dosage form, for the purpose of reducing mixing during operation of active agent between layers in the osmotic dosage form. No mention is made of how to obtain fast release of a drug from the osmotic dosage forms.

Also of note are U.S. Pat. Nos. 5,169,638; 5,536,507; 4,892,778; and 4,940,465. Again, no mention is made of how to obtain fast release of a drug from the dosage forms disclosed in these patents.

Accordingly, there remains a need for dosage forms and methods that combine fast release of a drug together with controlled release of a same or different drug from the same dosage form.

### SUMMARY OF THE INVENTION

In an aspect, the invention relates to an osmotic dosage form comprising: a semi-permeable membrane; a lubricating subcoat located within the semi-permeable membrane; an orifice in the semi-permeable membrane located at an end of the semi-permeable membrane; a drug layer located adjacent to the orifice and within the lubricating subcoat; a push layer located within the lubricating subcoat and on a side of the drug layer opposite from the orifice; wherein an area of the orifice is greater than or equal to about 1,600 mil²; and wherein the drug layer comprises from about 20 wt % to about 90 wt % microcrystalline cellulose, and less than or equal to about 10 wt % of a drug, based on the total weight of the drug layer.

In another aspect, the invention relates to a method of making an osmotic dosage form comprising: providing a semi-permeable membrane; providing a lubricating subcoat located within the semi-permeable membrane; locating an orifice in the semi-permeable membrane at an end of the semi-permeable membrane; locating a drug layer adjacent to the orifice and within the lubricating subcoat; locating a push layer within the lubricating subcoat and on a side of the drug layer opposite from the orifice; wherein an area of the orifice is greater than or equal to about 1,600 mil²; and wherein the drug layer comprises from about 20 wt % to about 90 wt % microcrystalline cellulose, and less than or equal to about 10 wt % of a drug, based on the total weight of the drug layer.

### BRIEF DESCRIPTION OF THE DRAWINGS

FIG. 1 shows a bi-layer osmotic dosage form according to the invention.

FIG. 2 shows a tri-layer osmotic dosage form according to the invention.

FIGS. 3-8 shows in vitro release plots for dosage forms according to the invention.

FIG. 9 shows the impact of orifice configuration on onset of delivery.

### DETAILED DESCRIPTION

### I. Introduction

The inventors have unexpectedly discovered that it is possible to address the problems noted above in the art using an osmotic dosage form comprising a semi-permeable membrane; a lubricating subcoat; an orifice in the semi-permeable membrane located at an end of the semi-permeable membrane; a drug layer located adjacent to the orifice and within the semi-permeable membrane; a push layer located within the semi-permeable membrane and on a side of the drug layer opposite from the orifice; wherein an area of the orifice is greater than or equal to about 1,600 mil²; and wherein the drug layer comprises from about 20 wt % to about 90 wt % microcrystalline cellulose, and less than or equal to about 10 wt % of a drug.

In particular, the inventive dosage form and methods provide for fast release of a drug together with controlled release of a same or different drug from the same dosage form. This is accomplished using specific compositions not previously defined in the art that provide the unexpected and desired benefit. The inventive dosage form and methods can reduce or eliminate the needs for immediate release overcoats, while still providing good controlled delivery profiles. Another benefit of the inventive dosage form and methods is that incorporation of a push layer promotes good delivery profiles for prolonged periods, while still providing fast release of drug. The performance improvements (fast release coupled with controlled release) are detailed in the Examples below.

The invention, and embodiments thereof, will now be described in more detail.

### II. DEFINITIONS

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Controlled release” or “controllably releasing” means continuous release or continuously releasing of a drug or a dose of a drug over a prolonged period.

“Dosage form” means a drug in a medium, carrier, vehicle, or device suitable for administration to a patient.

“Drug” means a pharmaceutically active agent or a pharmaceutically acceptable salt thereof. Drugs useful in the practice of this invention include, but are not limited to, the following: prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, topiramate, paliperidone, oxybutynin, methyl phenidate, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-varies hydroxyprogesterone acetate, 19-nor-progesterone, norgestrel, norethindrone, norethisterone, norethiederone, progesterone, norgesterone, norethynodrel, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, capropril, mando, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, imipramine, and terazosine HCl di-hydrate. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, prolactin, somatostatin, lypressin, pancreozymin, luteinizing hormone, LHRH, LHRH agonists and antagonists, leuprolide, interferons, interleukins, growth hormones such as human growth hormone, bovine growth hormone and porcine growth hormone, fertility inhibitors such as the prostaglandins, fertility promoters, growth factors, coagulation factors, human pancreas hormone releasing factor, analogs and derivatives of these compounds, and pharmaceutically acceptable salts of these compounds or their analogs or derivatives, and various combinations of these compounds, and various combinations of these compounds with various pharmaceutically acceptable salts of the above compounds. “Different drugs” means drugs with substantially different chemical structures. “Substantially identical drugs” means drugs with substantially similar chemical structures. “Identical drugs” means drugs with identical chemical structures. In an embodiment, various drugs may be combined within a drug layer, including polymorphs of the identical drug.

“Drug layer” means that portion or portions of a dosage form that comprise a drug and from which the drug is controllably released. In an embodiment, the drug layer is located within the lubricating subcoat.

“Lubricating subcoat” means a layer or film that is water-soluble, or partially water-soluble, and is permeable to the passage of an external fluid, such as water and/or biological fluids, and acts as a lubricating layer for the smooth expansion of the hydrating push layer, which displaces the dissolving drug layer(s). Materials useful for forming the lubricating subcoat are essentially hydrophilic and provide lubricity when hydrated. Examples of materials and methods for forming the lubricating subcoat, and structures of the osmotic dosage forms that comprise the lubricating subcoat, are disclosed elsewhere herein. In an embodiment, the lubricating subcoat is located within the semi-permeable membrane.

“Oral” means suitable for oral administration, when used to describe a dosage form.

“Orifice” means a hole or passageway formed through the semi-permeable membrane of an osmotic dosage form. Examples of exit ports and methods of making them that are useful in the practice of this invention are presented elsewhere herein. In an embodiment, an area of the orifice is greater than or equal to about 1,600 mil², more preferably greater than or equal to 6,300 mil², and most preferably greater than or equal to 17,000 mil².

“Osmagent” means a material that establishes an osmotic activity gradient across the semi-permeable membrane. Exemplary osmagents include salts, such as sodium chloride, potassium chloride, lithium chloride, etc. and sugars, such as raffinose, sucrose, glucose, lactose, and carbohydrates.

“Osmotic dosage form” means dosage forms that, in general, utilize osmotic pressure to generate a driving force for imbibing fluid into a compartment formed, at least in part, by a semi-permeable membrane that permits free diffusion of fluid but not drug. Examples of osmotic dosage forms useful in the practice of this invention are presented elsewhere herein.

“Patient” means an animal, preferably a mammal, more preferably a human, in need of therapeutic intervention.

“Prolonged period” means a continuous period of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Push layer” means a displacement composition that is positioned within the osmotic dosage form such that as the push layer expands during use, the materials forming the controlled release drug layer are expelled from the osmotic dosage form via the first orifice and/or one or more additional orifices located in the semi-permeable membrane adjacent to the first orifice. The push layer can be positioned in contacting layered arrangement with the controlled release drug layer or can have one or more intervening layers separating the push layer and drug layer. In an embodiment, the push layer is located within the lubricating subcoat and on a side of the drug layer opposite from the orifice. The push layer comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the lubricating subcoat and the semi-permeable membrane, the osmopolymer(s) swell and push against the drug layer to cause release of the drug from the dosage form via the orifice(s).

“Semi-permeable Membrane” or “Membrane” means a membrane that is permeable to the passage of an external fluid, such as water and/or biological fluids, but is substantially impermeable to the passage of components such as active pharmaceutical ingredients. Materials useful for forming the semi-permeable membrane are essentially non-erodible and are substantially insoluble in biological fluids during the life of a dosage form that comprises the semi-permeable membrane. Examples of materials and methods for forming the semi-permeable membrane, and structures of osmotic dosage forms that comprise the semi-permeable membrane are disclosed elsewhere herein.

### I. Osmotic Dosage Forms According to the Invention

Osmotic dosage forms are known generally in the art. Osmotic dosage forms typically utilize osmotic pressure as a driving force for imbibing fluid into a compartment formed, at least in part, by a semi-permeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

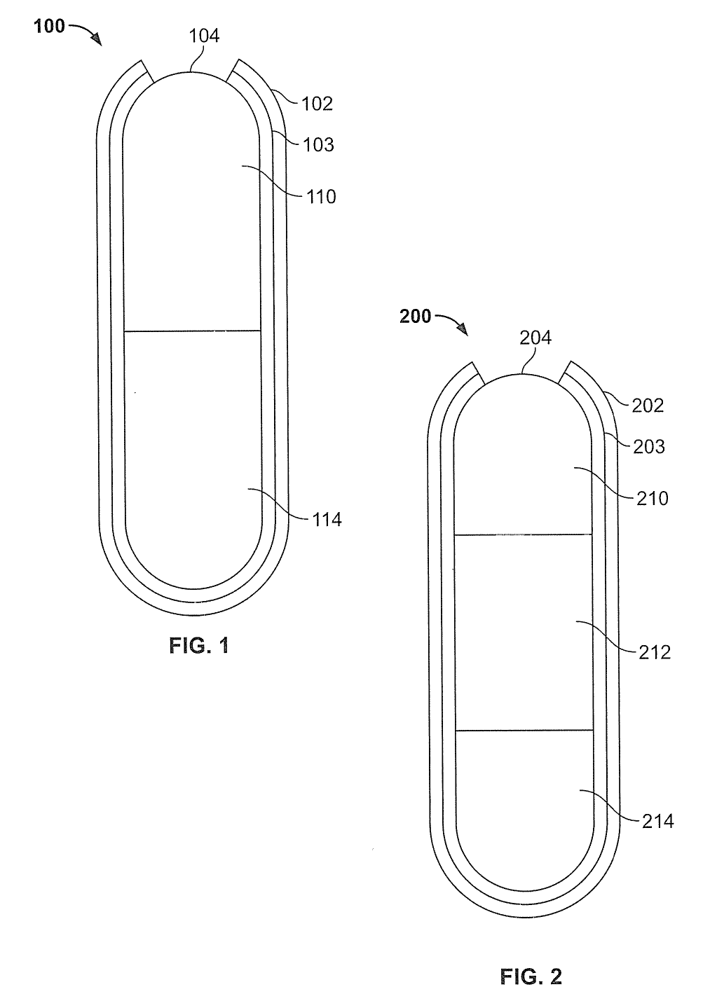

FIG. 1 shows a bi-layer osmotic dosage form according to the invention. Shown is osmotic dosage form 100, which comprises semi-permeable membrane 102, lubricating subcoat 103, orifice 104, drug layer 110, and push layer 114. Located within semi-permeable membrane 102 and lubricating subcoat 103 are drug layer 110 and push layer 114. Orifice 104 is located in lubricating subcoat 103 and semi-permeable membrane 102 at an end of lubricating subcoat 103 and semi-permeable membrane 102. This is a preferred embodiment with orifice 104 comprising an orifice in semi-permeable membrane 102 being coupled to an orifice in lubricating subcoat 103. In other embodiments, orifice 104 may penetrate only semi-permeable membrane 102. An area of orifice 104 is greater than or equal to about 1,600 mil², more preferably greater than or equal to 6,300 mil², and most preferably greater than or equal to 17,000 mil². Drug layer 110 is located adjacent to orifice 104 and within lubricating subcoat 103. Drug layer 110 comprises from about 40 wt % to about 90 wt % microcrystalline cellulose, preferably from about 24 wt % to about 44 wt % microcrystalline cellulose, and less than or equal to about 10 wt % of a drug, based on the total weight of drug layer 110. Further materials and methods used to formulate drug layer 110 are addressed herein. Push layer 114 is located within lubricating subcoat 103 and on a side of drug layer 110 opposite from orifice 104.

In operation, an osmotic gradient across semi-permable membrane 102 due to the presence of osmotically-active agents in drug layer 110 and/or push layer 114 causes fluid from the environment of use to be imbibed through semi-permable membrane 102 and lubricating subcoat 103, resulting in hydration of drug layer 110, and formation of a deliverable drug formulation (e.g., a solution, suspension, slurry or other flowable composition) within hydrating lubricating subcoat 103 and semi-permable membrane 102. The deliverable drug formulation is released through orifice 104 as fluid continues to enter through semi-permeable membrane 102. Push layer 114 operates by swelling (due to presence of osmotically active components contained in push layer 114) and pushing the deliverable drug formulation out through orifice 104. Even as drug formulation is released from dosage form 100, fluid continues to enter through semi-permeable membrane 102 and lubricating subcoat 103, thereby driving continued release through the combined action of drug layer 110 and push layer 114. In this manner, the drug in drug layer 110 is released in a sustained and continuous manner over a prolonged period.

Compared to conventional osmotically-controlled dosage forms, the onset of delivery from inventive dosage forms (e.g. dosage forms 100 and 200) may be unexpectedly hastened by increasing amounts of microcrystalline cellulose (MCC) and/or the orifice diameter. More particularly, as MCC levels in the drug layer are increased from about 20 wt % to about 90 wt %, preferably from about 24% to about 44 wt %, based on total weight of the drug layer, and as orifice diameter in the dosage form is increased from 45-mil to 90-mil to 150-mil, marked decreases in start-up time are noted. Moreover, at similar MCC levels, drug level does not appear to substantially affect in vitro functionality, as measured by USP Type VII dissolution testing.

FIG. 2 shows a tri-layer osmotic dosage form according to the invention.

Shown is osmotic dosage form 200, which comprises semi-permeable membrane 202, lubricating subcoat 203, orifice 204, first drug layer 210, second drug layer 212, and push layer 214. Located within lubricating subcoat 203 are first drug layer 210, second drug layer 212, and push layer 214. Orifice 204 is located in lubricating subcoat 203 and semi-permeable membrane 202 at an end of lubricating subcoat 203 and semi-permeable membrane 202. This is a preferred embodiment with orifice 204 comprising an orifice in semi-permeable membrane 202 being coupled to an orifice in lubricating subcoat 203. In other embodiments, orifice 204 may penetrate only semi-permeable membrane 202. An area of orifice 204 is greater than or equal to about 1,600 mil², and more preferably greater than or equal to 6,300 mil², and most preferably greater than or equal to 17,000 mil². First drug layer 210 is located adjacent to orifice 204 and within lubricating subcoat 203. First drug layer 210 comprises from about 20 wt % to about 90 wt % microcrystalline cellulose, preferably from about 24 wt % to about 44 wt % microcrystalline cellulose, and less than or equal to about 10 wt % of a drug, based on the total weight of first drug layer 210. Second drug layer 212 is located within lubricating subcoat 203, and between first drug layer 210 and push layer 214. Second drug layer 212 comprises from about 0 wt % to about 90 wt % microcrystalline cellulose, preferably from about 0 wt % to about 44 wt % microcrystalline cellulose, and less than or equal to about 50 wt % of a drug, based on the total weight of second drug layer 212. Further materials and methods used to formulate first drug layer 210 and second drug layer 212 are addressed herein. Push layer 214 is located within lubricating subcoat 203 and on a side of second drug layer 212 opposite from orifice 204.

In operation, an osmotic gradient across semi-permeable membrane 202 due to the presence of osmotically-active agents in first drug layer 210 and/or second drug layer 212 and/or push layer 214 causes fluid from the environment of use to be imbibed through semi-permeable membrane 202 and lubricating subcoat 203. This results in hydration of first drug layer 210 and second drug layer 212, and formation of a deliverable drug formulation (e.g., a solution, suspension, slurry or other flowable composition) within hydrated lubricating subcoat 203 and semi-permeable membrane 202. The deliverable drug formulation is released through orifice 204 as fluid continues to enter through semi-permeable membrane 202 and lubricating subcoat 203. Push layer 214 operates by swelling (due to presence of osmotically active components contained in push layer 214) and pushing the deliverable drug formulation out through orifice 204. Even as drug formulation is released from dosage form 200, fluid continues to enter through semi-permeable membrane 202 and lubricating subcoat 203, thereby driving continued release through the combined action of first drug layer 210, second drug layer 212, and push layer 214. In this manner, the drug(s) in first drug layer 210 and second drug layer 212 is/are released in a sustained and continuous manner over a prolonged period. Ascending drug release profiles can be obtained by providing a higher drug loading in second drug layer 212 than first drug layer 210. Other drug release profiles may also be achievable by varying amount of drug(s) in first drug layer 210 and second drug layer 212.

Osmotic dosage forms in accord with the present invention may be manufactured by standard techniques. For example, the osmotic dosage form may be manufactured by the wet granulation technique. In the wet granulation technique, materials making up the drug layer (e.g. 110 in FIG. 1 or 210 in FIG. 2) are blended using an organic solvent, such as denatured anhydrous ethanol, as the granulation fluid. Additional ingredients can be dissolved in a portion of the granulation fluid, and this latter prepared solution may be slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, stearic acid, and/or another suitable lubricant, is added to the drug granulation, and the granulation is put into milling jars and mixed on a jar mill for up to 10 minutes. The composition is pressed into a layer, for example, in a Manesty® press or a Korsch multi-layer press. The push layer may be prepared in similar fashion. For multi-layer cores according to the invention, the drug layer(s) is/are pressed into a mold, followed by the push layer. These intermediate compression steps typically take place under a force of about 50-100 newtons. Final stage compression typically takes place at a force of 3500 newtons or greater, often 3500-5000 newtons or more, depending on core diameter.

Pan coating may be conveniently used to provide the lubricating subcoat and semi-permeable membrane of the completed dosage form. In the pan coating system, the lubricating subcoat and semi-permeable membrane compositions are deposited by successive spraying of the appropriate lubricating subcoat or semi-permeable membrane composition onto the compressed multi-layered core, accompanied by tumbling in a rotating pan. A pan coater is used because of its availability at commercial scale. Other techniques can be used for coating the compressed core. Once coated and drilled, the coated cores are dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the lubricating subcoat or semi-permeable membrane of the osmotic dosage form may be formed in one technique using the air-suspension procedure. This procedure consists of suspending and tumbling the compressed core in a current of warmed air until the lubricating subcoat or semi-permeable membrane compositions are applied to the core. The air-suspension procedure is well suited for independently forming the lubricating subcoat or semi-permeable membrane of the osmotic dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The osmotic dosage form also can be coated with a Wurster® air-suspension coater using, for example, methylene dichloride blended with methanol as a cosolvent for the semi-permeable membrane forming material. An Aeromatic® air-suspension coater can be used employing a cosolvent.

Additional layers, besides the lubricating subcoat and semi-permeable membrane, may be coated on the osmotic dosage form. Certain additional layers may be known conventionally. For instance, optional water-soluble overcoats, which may be colored (e.g., Opadry colored coatings) or clear (e.g., Opadry Clear), may be coated on the osmotic dosage form to provide the finished dosage form.

In an embodiment, an orifice may be drilled in the ends of the osmotic dosage form. In an embodiment, the orifice(s) are formed or formable from a substance or polymer that erodes, dissolves or is leached from the outer wall to thereby form an exit orifice. The substance or polymer may include, for example, an erodible poly(glycolic) acid or poly(lactic) acid in the semi-permeable membrane; a gelatinous filament; a water-removable poly(vinyl alcohol); a leachable compound, such as a fluid removable pore-former selected from the group consisting of inorganic and organic salt, oxide and carbohydrate. The orifice, or a plurality of orifices, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice. The exit can have any shape, such as round, triangular, square, elliptical and the like for the uniform metered dose release of a drug from the dosage form. The osmotic dosage form can be constructed with one or more orifices in spaced-apart relation or one or more surfaces of the dosage form. Drilling, including mechanical and laser drilling, through the semi-permeable membrane and lubricating subcoat can be used to form the orifice(s). Such orifice(s)and equipment for forming such orifice(s)are disclosed in U.S. Pat. No. 3,916,899, by Theeuwes and Higuchi and in U.S. Pat. No. 4,088,864, by Theeuwes, et al.

While there has been described and pointed out features and advantages of the invention, as applied to present embodiments, those skilled in the medical art will appreciate that various modifications, changes, additions, and omissions in the method described in the specification can be made without departing from the spirit of the invention. In particular, it is not intended that the invention be limited in any way to the scope of the following exemplary embodiments.

### IV. EXAMPLES

### Example 1

### 8.4 mg System with Varying MCC Content

Paliperidone longitudinally shaped bilayer 8.4 mg systems were manufactured as follows: paliperidone, polyethylene oxide with average molecular weight of 200,000, microcrystalline cellulose and cross-linked povidone with average molecular weight of more than 1,000,000 (PVP XL), and polyvinylpyrrolidone (Povidone K29-32) were added to a glass jar. Next, the dry materials were mixed for 30 seconds. Then, approximately 10 ml of denatured anhydrous alcohol was slowly added to the blended materials with continuous mixing for approximately 2 minutes. Next, the freshly prepared wet granulation was passed through a 16-mesh sieve, then allowed to dry at room temperature for approximately 18 hours, and again passed through a 16-mesh screen. Next, the granulation was transferred to an appropriate container and granulation was then lubricated with stearic acid and magnesium stearate. The following granulations were manufactured at beaker scale (10 g). The polyethylene oxide/microcrystalline cellulose ratio was varied to determine the effect on functionality. Table 1 describes the formulation for the 8.4 mg system.

TABLE 1

| | Granulation ID | | |
|---|---|---|---|
| Material | PS-04 | PS-05 | PS-06 |
| Paliperidone | 5.00% | 5.00% | 5.00% |
| Polyethylene oxide | 42.50% | 25.50% | 0.00% |
| N-80 | | | |
| Microcrystalline Cellulose | 42.50% | 59.50% | 85% |
| Povidone (K29-32) | 4.00% | 4.00% | 4.00% |
| Stearic Acid | 0.50% | 0.50% | 0.50% |
| Magnesium Stearate | 0.50% | 0.50% | 0.50% |
| Crospovidone XL | 5.00% | 5.00% | 5.00% |

Next, a push composition was prepared as follows: first, a binder solution was prepared. Polyvinylpyrrolidone identified as K29-32 having an average molecular weight of 40,000 was dissolved in water. Then, sodium chloride and ferric oxide were sized using a Quadro Comil with a 21-mesh screen. Then, the screened materials and Polyethylene oxide (approximately 7,000,000 molecular weight) were added to a fluid bed granulator bowl. The dry materials were fluidized and mixed while binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with butylated hydroxytoluene and lubricated with magnesium stearate. Table 2 shows the composition the push layer granulation.

TABLE 2

| | Code | |
|---|---|---|
| Component | Number | Wt % |
| Polyetheylene Oxide | 0009567 | 63.67 |
| 7000 K, TG, LEO | | |
| NaCl | 0000476 | 30.00 |
| HPMC-E5 | 0001634 | 5.00 |
| Red Ferric Oxide | 0006064 | 1.00 |
| Magnesium Stearate | 0001161 | 0.25 |
| BHT | 0002652 | 0.08 |

### Example 2

### 2 mg System with Varying MCC Content

Paliperidone longitudinally shaped bilayer 2 mg systems were manufactured as follows: paliperidone, polyethylene oxide with average molecular weight of 200,000, microcrystalline cellulose and cross-linked povidone with average molecular weight of more than 1,000,000 (PVP XL), and polyvinylpyrrolidone (Povidone K29-32) were added to a glass jar. Next, the dry materials were mixed for 30 seconds. Then, approximately 10 ml of denatured anhydrous alcohol was slowly added to the blended materials with continuous mixing for approximately 2 minutes. Next, the freshly prepared wet granulation was passed through a 16-mesh sieve, then allowed to dry at room temperature for approximately 18 hours, and passed again through a 16-mesh screen. Next, the granulation was transferred to an appropriate container and granulation was then lubricated with stearic acid and magnesium stearate. The following granulations were manufactured at beaker scale (10 g). The polyethylene oxide/microcrystalline cellulose ratio was varied to determine the effect on functionality. Table 3 describes the formulation for the 2 mg system.

TABLE 3

| | Granulaion ID | | |
|---|---|---|---|
| Material | PS-09 | PS-10 | PS-11 |
| Paliperidone | 1.20% | 1.20% | 1.20% |
| Polyethylene Oxide | 44.80% | 54.80% | 64.80% |
| N-80 | | | |
| Microcrystalline | 44.00% | 34.00% | 24.0% |
| Cellulose | | | |
| Povidone (K29-32) | 4.00% | 4.00% | 4.00% |
| Stearic Acid | 0.50% | 0.50% | 0.50% |
| Magnesium Stearate | 0.50% | 0.50% | 0.50% |
| Crosspovidone XL | 5.00% | 5.00% | 5.00% |

Next, a push composition was prepared as follows: first, a binder solution was prepared. Polyvinylpyrrolidone identified as K29-32 having an average molecular weight of 40,000 was dissolved in water. Then, sodium chloride and ferric oxide were sized using a Quadro Comil with a 21-mesh screen. Then, the screened materials and Polyethylene oxide (approximately 7,000,000 molecular weight) were added to a fluid bed granulator bowl. The dry materials were fluidized and mixed while binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with butylated hydroxytoluene and lubricated with magnesium stearate. Table 4 shows the composition the push layer granulation.

TABLE 4

| | Code | |
|---|---|---|
| Component | Number | Wt % |
| Polyetheylene Oxide 7000 K, | 0009567 | 63.67 |
| TG, LEO | | |
| NaCl | 0000476 | 30.00 |
| HPMC-E5 | 0001634 | 5.00 |
| Red Ferric Oxide | 0006064 | 1.00 |
| Magnesium Stearate | 0001161 | 0.25 |
| BHT | 0002652 | 0.08 |

Next, the drug and push compositions of either Example 1 or Example 2 were compressed into bilayer tablets on the Carver Tablet Press. First, 167 mg of a drug composition of either Example 1 or Example 2 was added to the die cavity and pre-compressed, then, 111 mg of the push composition was added and the layers were pressed under a pressure head of approximately 0.5 metric ton into a 3/16″ (0.476 cm) diameter bilayer longitudinal arrangement.

The bilayered arrangements were coated with a lubricating subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol to make an 8% solids solution. The wall-forming composition was sprayed onto and around the bilayered arrangements in a pan coater until approximately 20 mg of laminate is applied to each tablet.

The bilayered, subcoated cores were coated with a semi-permeable wall. The wall forming composition comprised 99% cellulose acetate having a 39.8% acetyl content and 1% polyethylene glycol comprising a 3,350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 55 mg of membrane was applied to each tablet.

Next, a 145-mil (3.7 mm) exit passageway was drilled through the semi-permeable wall to connect the drug layer with the exterior of the dosage system. The residual solvent was removed by drying for 72 hours as 45° C. and 45% humidity.

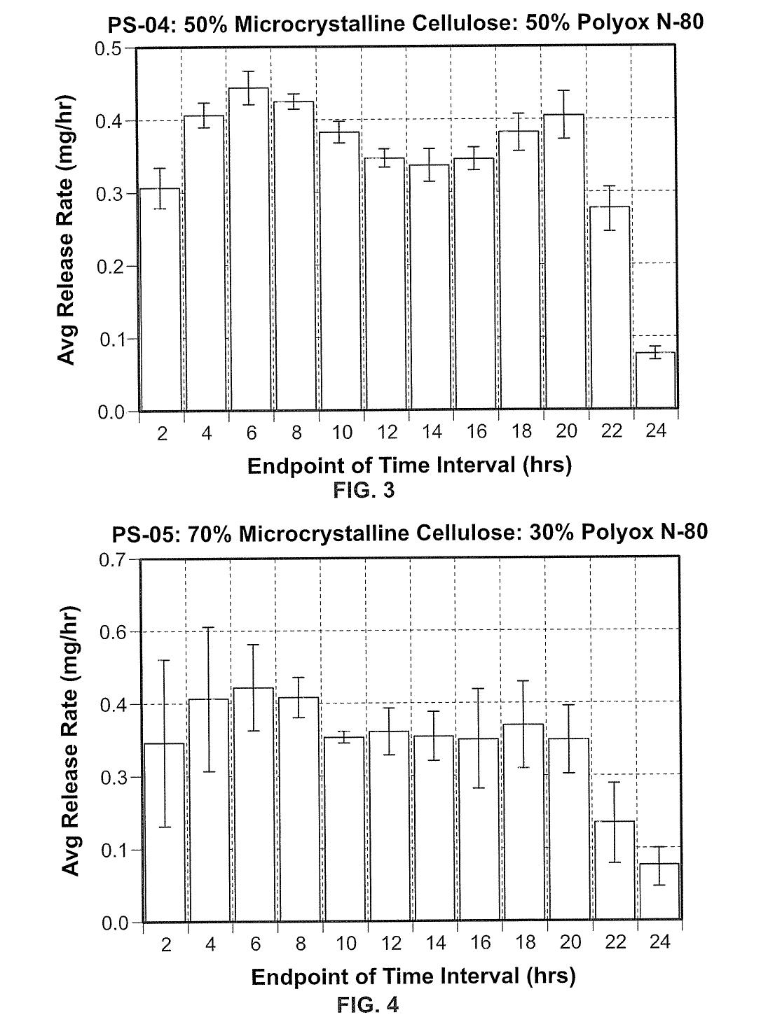

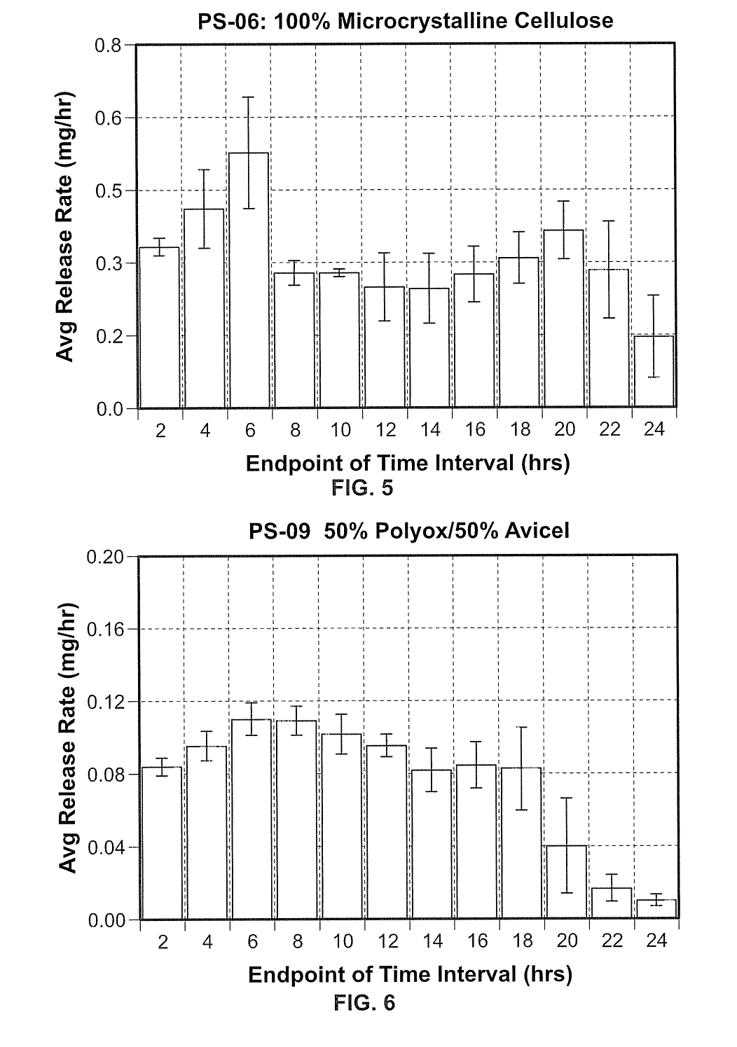

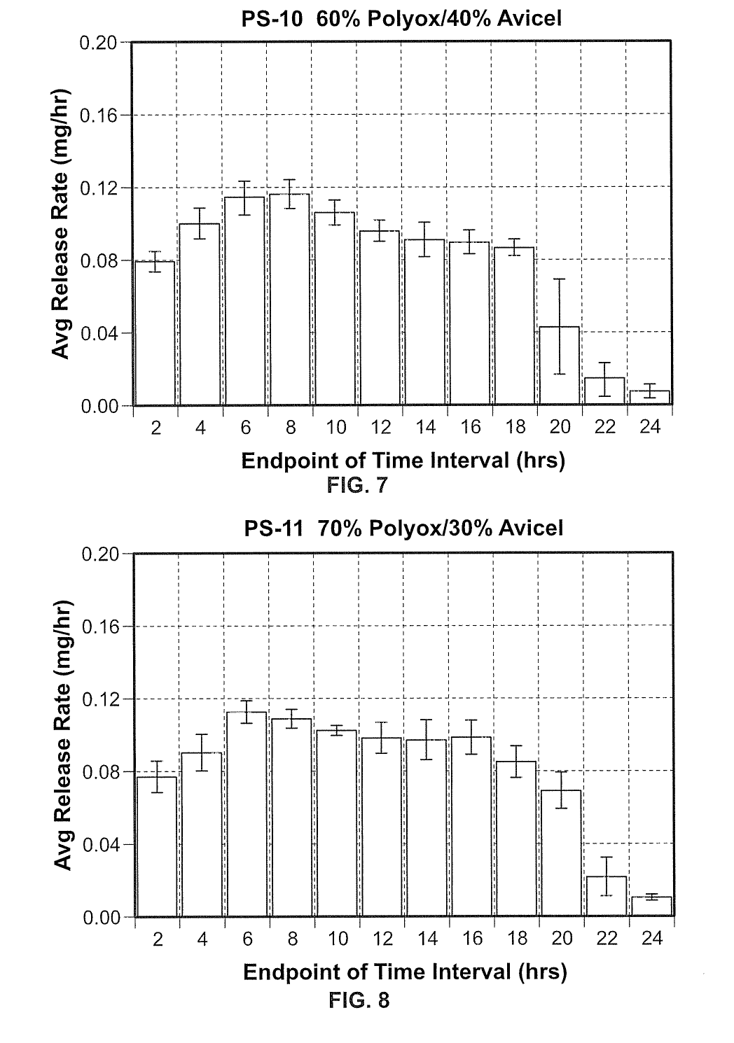

The dosage form produced by this manufacture was designed to deliver either 8.4 mg or 2 mg of paliperidone in a controlled delivery pattern from the drug-containing core. FIG. 1 shows a system configuration. Systems were released according to the Type VII USP dissolution bath resulting in the release profiles in FIGS. 3-8.

Table 5 and FIGS. 3-5 show the release rate characteristics of the system made using the drug composition of Example 1. Results show that the systems containing a 1:1 Polyethylene Oxide: Microcrystalline Cellulose (PS-04) ratio achieved a better zero-order release rate profile compared to the other two formulations. This platform has a start-up time of less than one hour with a duration of 20 hrs and substantially smaller values for the variability within and between systems though rapid start up is achieved across the range of MCC level, more variable release rates are noted at 60% and 85% MCC.

TABLE 5

| | | | Residual | | 90% of drug | | Coefficient of |
|---|---|---|---|---|---|---|---|
| | | Start-Up | Drug | Mass | delivered | Coefficient of | Variation |
| | Subcoat | Time | (% of Mass | Balance | (t90) | Variation Within | Between |
| Core ID | Weight | (hour) | Balance) | (mg) | (hour) | (%) | (%) |
| PS-04 | 18.8 | 0.3 | 2.82 | 8.4 | 20.4 | 6.9 | 2.3 |
| PS-05 | 17.7 | 0.6 | 4.67 | 8.5 | 20.6 | 37.8 | 13.5 |
| PS-06 | 17.3 | 0.0 | 3.65 | 8.2 | 21.3 | 25.4 | 4.0 |

While higher concentrations of microcrystalline cellulose in the formulation were explored for the 8.4 mg systems, lower amounts of microcrystalline cellulose were explored for the 2 mg system. Table 6 and FIGS. 6-8 shows the release rate characteristics of the system made using the drug composition of Example 2. As suggested by the data, start-up time decreases with increasing MCC content, while release rate variability is largely unaffected. Similar functionality is achieved across the preferable MCC range (24-44%) studied.

TABLE 6

| | | | Residual | | 90% of drug | | Coefficient of |
|---|---|---|---|---|---|---|---|
| | | Start-Up | Drug | Mass | delivered | Coefficient of | Variation |
| | Subcoat | Time | (% of Mass | Balance | (t90) | Variation Within | Between |
| Core ID | Weight | (hour) | Balance) | (mg) | (hour) | (%) | (%) |
| PS-09 | 22.1 | 0.5 | 1.14 | 1.9 | 17.6 | 4.2 | 8.1 |
| PS-10 | 22.6 | 0.6 | 1.05 | 1.9 | 17.6 | 3.1 | 3.8 |
| PS-11 | 22.4 | 0.8 | 1.25 | 1.9 | 18.4 | 7.1 | 6.2 |

The results suggest that when increasing the amount of microcrystalline cellulose to a total of about 90 wt % in the formulation the functionality of the system is affected. The release rate profile becomes erratic and more variable as the amount of microcrystalline cellulose is increased. However the zero-order profile is substantially unaffected when the preferred range of 24-44% of microcrystalline cellulose is incorporated in the formulation.

### Example 3

### 2 mg System with Varying Orifice Configuration

The number and size of the delivery orifice was varied in Formulation PS-10 from Example 2 to elucidate the effect on onset of delivery as measured by the amount of paliperidone released in the first 2 hours. Systems were manufactured per Example 2, except that the number and diameter of the delivery orifice(s) were specified as follows:

| Set ID | Description |
|---|---|
| 2 × 25-mil | two 25-mil diameter orifices drilled in drug dome |
| 1 × 45-mil | one 45-mil diameter orifice drilled in drug dome |
| 1 × 60-mil | one 60-mil diameter orifice drilled in drug dome |
| 1 × 90-mil | one 90-mil diameter orifice drilled in drug dome |
| 1 × 150-mil | one 150-mil diameter orifice drilled in drug dome |

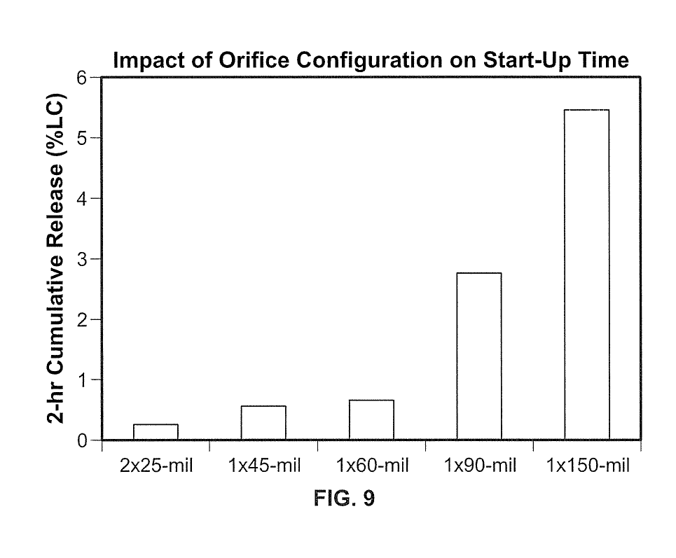

Samples from each ID set were tested for release rate per the method described in Examples 1 and 2. The effect of orifice configuration on onset of delivery is exhibited in FIG. 9. For rapid start up, 1×45-mil and 1×60-mil are preferable to 2×25-mil. In turn, 1-90-mil is more preferable and 1×150-mil is most preferable.

What is claimed is:

1. An osmotic dosage form comprising:
a semi-permeable membrane;
a lubricating subcoat located within the semi-permeable membrane;
an orifice in the semi-permeable membrane located at an end of the semi-permeable membrane;
a drug layer located adjacent to the orifice and within the lubricating subcoat;
a push layer located within the lubricating subcoat and on a side of the drug layer opposite from the orifice;
wherein an area of the orifice is greater than or equal to about 1,600 mil²; and
wherein the drug layer comprises from about 20 wt % to about 90 wt % microcrystalline cellulose, and less than or equal to about 10 wt % of a drug, based on the total weight of the drug layer.

2. The osmotic dosage form of claim 1, further comprising an orifice in the lubricating subcoat coupled to the orifice in the semi-permeable membrane.

3. The osmotic dosage form of claim 1, wherein area of the orifice is greater than or equal to 6,300 mil².

4. The osmotic dosage form of claim 1, wherein area of the orifice is greater than or equal to 17,000 mil².

5. The osmotic dosage form of claim 1, wherein the drug layer comprises an osmagent.

6. The osmotic dosage form of claim 1, wherein the drug layer comprises from about 24 wt % to about 44 wt % microcrystalline cellulose, based on the total weight of the drug layer.

7. The osmotic dosage form of claim 1, wherein the drug layer comprises more than one drug.

8. The osmotic dosage form of claim 1, further comprising a second drug layer located within the lubricating subcoat, and between the drug layer and the push layer.

9. The osmotic dosage form of claim 8, wherein the second drug layer comprises from about 0 wt % to about 90 wt % microcrystalline cellulose, based on the total weight of the second drug layer.

10. The osmotic dosage form of claim 9, wherein the second drug layer comprises from about 0 wt % to about 44 wt % microcrystalline cellulose, based on the total weight of the second drug layer.

11. The osmotic dosage form of claim 8, wherein the second drug layer comprises less than or equal to about 50 wt % of a drug, based on the total weight of the second drug layer.

12. A method of making an osmotic dosage form comprising:
providing a semi-permeable membrane;
providing a lubricating subcoat located within the semi-permeable membrane;
locating an orifice in the semi-permeable membrane at an end of the semi-permeable membrane;
locating a drug layer adjacent to the orifice and within the lubricating subcoat;
locating a push layer within the lubricating subcoat and on a side of the drug layer opposite from the orifice;
wherein an area of the orifice is greater than or equal to about 1,600 mil²; and
wherein the drug layer comprises from about 20 wt % to about 90 wt % microcrystalline cellulose, and less than or equal to about 10 wt % of a drug, based on the total weight of the drug layer.

13. The method of claim 12, further comprising coupling an orifice in the lubricating subcoat to the orifice in the semi-permeable membrane.

14. The method of claim 12, wherein area of the orifice is greater than or equal to 6,300 mil².

15. The method of claim 12, wherein area of the orifice is greater than or equal to 17,000 mil².

16. The method of claim 12, wherein the drug layer comprises an osmagent.

17. The method of claim 12, wherein the drug layer comprises from about 24 wt % to about 44 wt % microcrystalline cellulose, based on the total weight of the drug layer.

18. The method of claim 12, wherein the drug layer comprises more than one drug.

19. The method of claim 12, further comprising a second drug layer located within the lubricating subcoat, and between the drug layer and the push layer.

20. The method of claim 19, wherein the second drug layer comprises from about 0 wt % to about 90 wt % microcrystalline cellulose, based on the total weight of the second drug layer.

21. The method of claim 20, wherein the second drug layer comprises from about 0 wt % to about 44 wt % microcrystalline cellulose, based on the total weight of the second drug layer.

22. The method of claim 19, wherein the second drug layer comprises less than or equal to about 50 wt % of a drug, based on the total weight of the second drug layer.
